(19) United States
(12) Patent Application Publication
(10) Pub. No.: US 2010/0056452 A1
(43) Pub. Date: Mar. 4, 2010

(54) ANGIOGENESIS-PROMOTING SUBSTRATE

(75) Inventors: Michael Ahlers, Eberbach (DE); Burkhard Schlosshauer, Tuebingen (DE); Lars Dreesmann, Langenau (DE)

Correspondence Address: LEYDIG VOIT & MAYER, LTD, 700 THIRTEENTH ST. NW, SUITE 300, WASHINGTON, DC, 20005-3960, US

(73) Assignee: GELITA AG, Eberbach (DE)

(21) Appl. No.: 12/615,549

(22) Filed: Nov. 10, 2009

Related U.S. Application Data
(63) Continuation of application No. PCT/EP2008/003895, filed on May 15, 2008.

(30) Foreign Application Priority Data
May 16, 2007 (DE) 10 2007 024 239.7

(51) Int. Cl. A61K 38/16; C07K 14/00; A61P 7/00; B32B 5/00

(52) U.S. Cl. 514/12; 530/354; 428/220; 428/332

(57) ABSTRACT

To provide an angiogenesis-promoting substrate which can be easily and cost-effectively produced, it is proposed that the substrate comprise a non-porous shaped body formed from a gelatin-containing material which is insoluble and resorbable under physiological conditions.

### CROSS-REFERENCE TO RELATED APPLICATIONS

This application is a continuation of international application number PCT/EP2008/003895 filed on May 15, 2008.

The present disclosure relates to the subject matter disclosed in international application number PCT/EP2008/003895 of May 15, 2008 and German application number 10 2007 024 239.7 of May 16, 2007, which are incorporated herein by reference in their entirety and for all purposes.

### BACKGROUND OF THE INVENTION

The present invention relates to an angiogenesis-promoting substrate.

In living mammals, endothelial cells which line existing blood vessels form new capillaries wherever these are required. The endothelial cells have the remarkable capability of adapting their number and arrangement to the local requirements. Tissues are dependent upon the blood supply which is provided by the blood vessel system. The vessel system, in turn, is dependent upon the endothelial cells. The endothelial cells create an adaptable life-ensuring system which branches into almost all regions of the body.

While the largest blood vessels, the arteries and veins, have a thick, strong wall of connective tissue and partly smooth muscles and are lined on the inside with only an extremely thin, single layer of endothelial cells, in the finest branches of the vessel system, the capillaries, walls are found which consist solely of endothelial cells and a so-called basal lamina. Endothelial cells thus line the entire blood vessel system running from the heart into the smallest capillary, and they control the passage of factors and cells into and out of the blood stream.

In the event of a deficiency of oxygen, tissue cells release angiogenic factors which activate the growth of new capillaries. Local (mechanical) irritations and infections also cause proliferation of new capillaries, most of which recede and disappear once the inflammation subsides.

The newly forming blood vessels first always develop as capillaries which sprout on existing small vessels. This process is called angiogenesis.

The sprouting of the capillaries propagates until the respective sprout encounters another capillary and can unite with it, so that blood can circulate therein (cf., for example, B. Alberts et al., Molekularbiologie der Zelle, VCH Weinheim, 3rd edition 1995, pages 1360-1364).

Factors which stimulate angiogenesis are widely known and include, for example, the factors HGF, FGF, VEGF and others.

In the literature (cf., for example, EP 1 415 633 A1 and EP 1 555 030 A1), administration of such angiogenesis-stimulating factors in a sustained release matrix was proposed, and a gelatin hydrogel comprising gelatin with an average molecular weight of 100,000 to 200,000 daltons (Da) was recommended as sustained release matrix.

The suitability of various types of collagen as scaffold in the formation of new vessels as well as their anti-angiogenetic effects are described. Reference is made to S. M. Sweeney et al., The Journal of Biological Chemistry, volume 278, No. 33, pages 30516 to 30524 (2003) and to R. Xu et al. in Biochemical and Biophysical Research Communications 289, pages 264 to 268 (2001) as examples of this literature.

The object underlying the present invention is to provide an angiogenesis-promoting substrate which can be manufactured easily and cost-effectively.

### SUMMARY OF THE INVENTION

This object is accomplished by an angiogenesis-promoting substrate comprising a non-porous shaped body formed from a gelatin-containing material which is insoluble and resorbable under physiological conditions.

Owing to their good biocompatibility, gelatin-based materials have been in use for quite some time for medical applications, for example, as matrix material for the release of pharmaceutically active substances or as carrier material for colonization with cells. In contrast, for example, to collagen, gelatin can be produced in reproducible quality and with a high degree of purity. Furthermore, it is essentially completely resorbable in the body.

Within the scope of the present invention, it has now, surprisingly, been found that the gelatin-containing material as such has an angiogenesis-promoting effect, i.e., stimulates the formation of new blood vessels in its immediate vicinity, without any further angiogenesis-promoting factors such as, for example, the aforementioned signalling molecules VEGF, FGF or HGV being required.

It is particularly remarkable that the angiogenesis-promoting effect in accordance with the invention is observed in a non-porous shaped body, which is formed from the gelatin-containing material. In earlier examinations conducted by the inventors, an angiogenesis-promoting effect was first found in porous shaped bodies made of gelatin-containing material, with the angiogenesis primarily taking place within the shaped bodies, i.e., an ingrowth of blood vessels into the pores, cavities or interspaces of the shaped body was observed. The pro-angiogenetic effect was, therefore, primarily attributed to the porous structure of the shaped body (see the German patent application with file number 10 2005 054 937). Examples of such structures are sponges, woven fabrics or fleeces.

In contrast to this, it has now been found that in accordance with the present invention a non-porous shaped body can also be used as angiogenesis-promoting substrate, with the blood vessel formation not taking place in the shaped body, but in its spatial environment. Without wishing to be bound to this theory, it is assumed that this effect is caused by a release of soluble components of the gelatin and, therefore, is substantially independent of the structure of the shaped body.

As a rule, non-porous shaped bodies made of a gelatin-containing material are easier to produce than those with a porous structure. On the other hand, use of a shaped body made of an insoluble material, which is only resorbed or broken down after a certain time, has in comparison with use of soluble or dissolved gelatin the advantage that the angiogenesis can be stimulated in a targeted manner at a certain location, namely in the vicinity of the shaped body used.

The gelatin-containing material is preferably a gelatin-based material and consists predominantly of gelatin. This means that the gelatin constitutes the largest proportion where further components are used in the material.

Further preferred is use of a gelatin-based material consisting essentially entirely of gelatin.

Particularly suitable gelatin types are pigskin gelatin, which is preferably high-molecular and has a Bloom value of approximately 160 to approximately 320 g.

To a considerably lesser extent, an angiogenesis-stimulating effect is also observed with low-molecular, water-soluble gelatin having an average molecular weight of less than 6 kDa, but such an effect is comparatively unspecific when compared with other agents that likewise stimulate to a lesser extent.

Therefore, the gelatin used preferably has an average molecular weight greater than approximately 6 kDa.

To ensure optimum biocompatibility of the substrate according to the invention in medical use, a gelatin having a particularly low content of endotoxins is preferably used as starting material. Endotoxins are products of metabolism or fractions of microorganisms which occur in the raw animal material. The endotoxin content of gelatin is indicated in international units per gram (I.U./g) and determined in accordance with the LAL test, the performance of which is described in the fourth edition of the European Pharmacopoeia (Ph. Eur. 4).

To keep the content of endotoxins as low as possible, it is advantageous to kill the microorganisms as early as possible in the course of the gelatin production. Furthermore, appropriate hygiene standards should be maintained during the manufacturing process.

The endotoxin content of gelatin can thus be drastically reduced by certain measures during the manufacturing process. These measures primarily include the use of fresh raw materials (for example, pigskin) with avoidance of storage times, thorough cleaning of the entire production plant immediately before start of the gelatin production and possibly exchange of ion exchangers and filter systems in the production plant.

The gelatin used within the scope of the present invention preferably has an endotoxin content of approximately 1,200 I.U./g or less, even more preferred approximately 200 I.U./g or less. Optimally, the endotoxin content lies at approximately 50 I.U./g or less, determined, in each case, in accordance with the LAL test. In comparison with this, many commercially available gelatins have endotoxin contents of over 20,000 I.U./g.

As indicated hereinabove, the non-porous shaped body of the angiogenesis-promoting substrate is formed in accordance with the invention from a material which is insoluble under physiological conditions, so that it maintains its structural integrity over a certain period of time, and the angiogenesis can be localized to the desired target area. However, since gelatin is dissolved quickly under physiological conditions, the gelatin-containing material is preferably cross-linked.

In accordance with a further embodiment of the present invention, a quick dissolution can be counteracted by using the gelatin together with other components which dissolve more slowly (examples of such resorbable biopolymers are chitosan and hyaluronic acid). Such components may be used for the purpose of temporary immobilization of the gelatin proportions.

If cross-linking is chosen for stabilization of the material, in particular, the gelatin proportion of the gelatin-containing material can be cross-linked, and chemical cross-linking or also enzymatic cross-linking can then be resorted to.

Preferred chemical cross-linking agents are aldehydes, dialdehydes, isocyanates, carbodiimides and alkyl dihalides. Formaldehyde, which simultaneously effects a sterilization of the shaped body, is particularly preferred.

The enzyme transglutaminase, which effects a linking of glutamine and lysine side chains of proteins, in particular, also of gelatin, is preferred as enzymatic cross-linking agent.

The stability with respect to resorption under the physiological conditions referred to hereinabove, to which the material is exposed during its use, can be simulated under corresponding standard physiological conditions in vitro. Here a PBS buffer (pH 7.2) is used at 37° C., and under these conditions the substrates can be tested and compared as to their time-dependent stability behavior.

The gelatin-containing material preferably has a prescribed degree of cross-linking. In particular, the resorption stability of the shaped body, i.e. the time during which it maintains its structural integrity under physiological conditions can be set by prescribing the degree of cross-linking. It is thus possible, for example, to use as angiogenesis-promoting substrates non-porous shaped bodies which in dependence upon the degree of cross-linking of the gelatin-containing material are stable for, for example, one, three, six or twelve weeks under physiological standard conditions, depending on for whatever period of time an angiogenetic effect is desired by the attending physician.

Surprisingly, it has also been found that the higher the degree of cross-linking of the gelatin-containing material, the higher is the angiogenesis-promoting effect of the shaped body, in particular, in the case of chemical cross-linking of the gelatin. This opens up further possibilities for also stimulating the angiogenesis in a quantitatively targeted manner.

The structure of the non-porous shaped body is preferably stabilized by a two-stage cross-linking, wherein at a first stage the gelatin-containing material in solution is subjected to a first cross-linking reaction, and a shaped body produced from this material is then further cross-linked at a second cross-linking stage.

Whereas at the first cross-linking stage, the cross-linking takes place in solution, in particular, a cross-linking in the gaseous phase, for example, using formaldehyde, is possible for the second cross-linking stage.

The production of shaped bodies from a gelatin-containing material by means of a two-stage cross-linking process is described in detail in the publication DE 10 2004 024 635 A1.

The two-stage cross-linking has, in particular, the advantage that overall a higher degree of cross-linking is obtainable, which, in addition, is then achievable substantially uniformly over the entire cross section of the shaped body. As a consequence of this, the degradation characteristics of the shaped body during the resorption are homogenous, so that it substantially maintains its structural integrity for the intended period of time in dependence upon the degree of cross-linking and is then completely resorbed in a relatively short time, whereby the structural integrity is lost.

Owing to the prescribed degree of cross-linking and the above-described homogenous degradation behavior, the angiogenesis-promoting effect of the substrate according to the invention can, therefore, be employed in a precisely targeted manner with respect to both time and space.

For many applications, the degree of cross-linking should be so selected that under the standard physiological conditions mentioned hereinabove approximately 20 wt % or less of the gelatin-containing material is broken down over 7 days.

The non-porous shaped body can be made with very different structures, which have not yet been discussed.

In accordance with a preferred embodiment of the invention, the shaped body is a sheet material. Sheet materials can be used in a variety of ways as medical substrates in or on the body.

It is particularly preferred for the shaped body to be a film. Such films can be produced in a simple way by casting a solution of a gelatin-containing material, and this process can be combined with the two-stage cross-linking process described hereinabove.

Films made from a gelatin-containing material are easy to handle and can be cut to the respectively required size by the attending physician. In order to increase the flexibility of the film, the gelatin-containing material can additionally contain one or more softeners. Preferred softeners are selected from glycerin, oligoglycerins, oligoglycols, sorbite and mannite.

The film preferably has a thickness ranging from approximately 20 to approximately 500 μm, further preferred from approximately 50 to approximately 100 μm.

In accordance with a further embodiment of the invention, the non-porous shaped body is in the form of particles. The particles can be, for example, globules, granulate or powder made from a gelatin-containing material.

Preferred particles have an average diameter of from approximately 0.1 mm to approximately 5 mm.

In accordance with an advantageous embodiment of the invention, the non-porous shaped body comprises one or more pharmaceutically active substances not based on gelatin. These can be, for example, anti-inflammatory and antibiotic agents.

In an embodiment of the invention, the non-porous shaped body is colonized with cells. In this case, the substrate according to the invention can be used for cell transplantations in which an angiogenesis is desired in the area of the implanted cells.

The present invention also relates to the use of a non-porous shaped body formed from a gelatin-containing material which is insoluble and resorbable under physiological conditions, for producing an angiogenesis-promoting substrate, which is intended for use in or on the body of a human being or an animal. Advantages and preferred embodiments of this use will be apparent, in particular, from the above description of the angiogenesis-promoting substrate according to the invention.

In a preferred use, the substrate is used as wound dressing or covering. By applying the substrate to injuries or burns, in particular, on the skin, the angiogenetic effect can contribute towards quicker wound healing.

In accordance with a further preferred embodiment, the angiogenesis-promoting substrate is intended for implantation in the body. Here the substrate can be intracorporally inserted at many different locations of the body, wherever a targeted promotion of the angiogenesis is required or desired.

Preferred areas of application of the angiogenesis-promoting substrate according to the invention are, for example, transplantations, the treatment of diabetes or of infarctions.

When performing therapeutic procedures using the angiogenesis-promoting substrate according to the invention, a non-porous shaped body is made available in the respectively required shape and size or is cut to size by the attending physician, in order to then be inserted into or placed on the corresponding area on the human or animal body.

These and further advantages of the invention are explained in detail hereinbelow with reference to the drawings and the examples.

### BRIEF DESCRIPTION OF THE DRAWINGS

FIG. 1: shows a photographic representation of the blood vessel formation without an angiogenesis-promoting substrate;

FIGS. 2a to 2c: show photographic representations of the blood vessel formation with various angiogenesis-promoting substrates according to the invention; and

FIG. 3: shows a photographic representation of the blood vessel formation after resorption of the angiogenesis-promoting substrate.

### DETAILED DESCRIPTION OF THE INVENTION

### Production of Films from a Gelatin-Containing Material

Gelatin films with three different degrees of cross-linking (films A, B and C) were produced by a two-stage cross-linking process as examples of non-porous shaped bodies.

For each of the three batches, 25 g of pigskin gelatin (300 g Bloom), 9 g of an 85 wt % glycerin solution and 66 g of distilled water were mixed, and the gelatin was dissolved at a temperature of 60° C. After degassing of the solutions by ultrasound, in order to perform the first cross-linking step, an aqueous formaldehyde solution (2.0 wt %, room temperature) was added, more specifically, 3.75 g of this solution for batch A, and 6.25 g of the solution for each of the batches B and C.

The mixtures were homogenized and spread with a doctor blade at approximately 60° C. in a thickness of approximately 250 μm onto a polyethylene base.

After drying at 30° C. and a relative atmospheric humidity of 30% for approximately one day, the films were detached from the PE base and redried for approximately 12 h under the same conditions. For performance of the second cross-linking step, the dried films (thickness approximately 50 μm) were exposed in a desiccator to the equilibrium vapor pressure of a 17 wt % aqueous formaldehyde solution at room temperature. In the case of films A and B, the duration of exposure to the formaldehyde vapor was 2 h, in the case of film C 17 h.

Of the shaped bodies produced in this way, film A has overall the lowest and film C overall the highest degree of cross-linking, film B lies between these. This is reflected in the different degradation behavior of the films, the resorption times of the described films under physiological conditions in tests on animals (see below) being between approximately 14 days (film A) and approximately 21 days (film C).

Owing to the use of glycerin as softener, the films exhibit adequate flexibility, in particular, in the hydrated state, to ensure easy handling during medical application, without having to fear that the films will break or tear.

### Proof of the Angiogenesis-Promoting Effect in Tests on Animals

The efficacy of the gelatin films A, B and C as angiogenesis-promoting substrates in vivo was examined in tests on animals. Ten-week-old mice of the Balb/C strain from the Charles River company (Sulzfeld) with a body weight of 20 g were used as test animals.

Pieces of the above-described gelatin films, each measuring 5×5 mm² were used as substrates. Two pieces of film having a certain degree of cross-linking were implanted subcutaneously in the neck area of each of the mice. To do so, the animals were anaesthetized and their coat was shaved off in the neck area. A piece of the neck skin was lifted with tweezers and an incision of approximately 1 cm in length was made. Through this incision, a subcutaneous pocket was created with blunt scissors, and, in each case, two pieces of film were placed in it with tweezers. The wound was closed with two single button knots.

After 12 days the animals were killed, and the angiogenetic effect of the implanted substrates was optically evaluated.

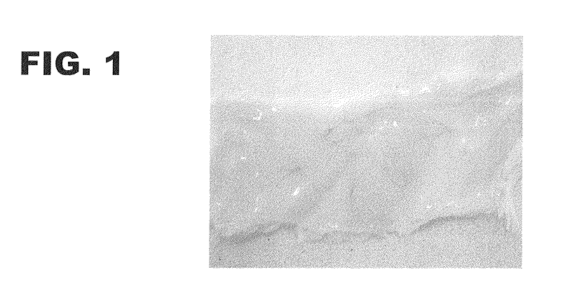

FIG. 1 shows as negative control the corresponding area of the subcutaneous tissue of a mouse in which no implantation of the angiogenesis-promoting substrate was performed. Only a very slight permeation with blood vessels is to be observed, as is normal for the subcutaneous tissue of the mouse.

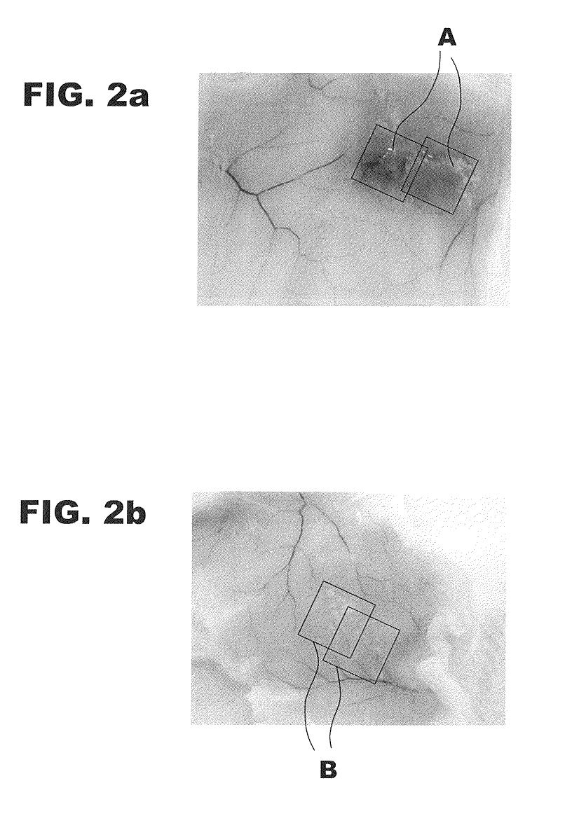

FIGS. 2a to 2c show photographs of the subcutaneous tissue in the area of the implanted pieces of film A, B and C, respectively, after the corresponding mice were killed 12 days after the implantation. The position of the pieces of film is marked by black squares (references A, B and C, respectively, for the corresponding film), as the films themselves are difficult to discern in the photograph. Experimentally, the films were partly dyed with Coomassie Brilliant Blue, as is apparent in FIG. 2a.

In all three representations, a significantly increased blood vessel formation is recognizable in the vicinity of the implanted pieces of film. Both the number and the size of the blood vessels are significantly higher in each case than in the negative control in FIG. 1. This result proves that the angiogenesis can be stimulated locally by non-porous shaped bodies formed from a gelatin-containing material which is insoluble and resorbable under physiological conditions.

In order to examine the time frame of the angiogenesis-promoting effect, two pieces of film of film B (middle degree of cross-linking) were implanted (as described above) in a further mouse. This mouse was killed after 21 days, and the subcutaneous tissue was optically evaluated again in the area of the implants.

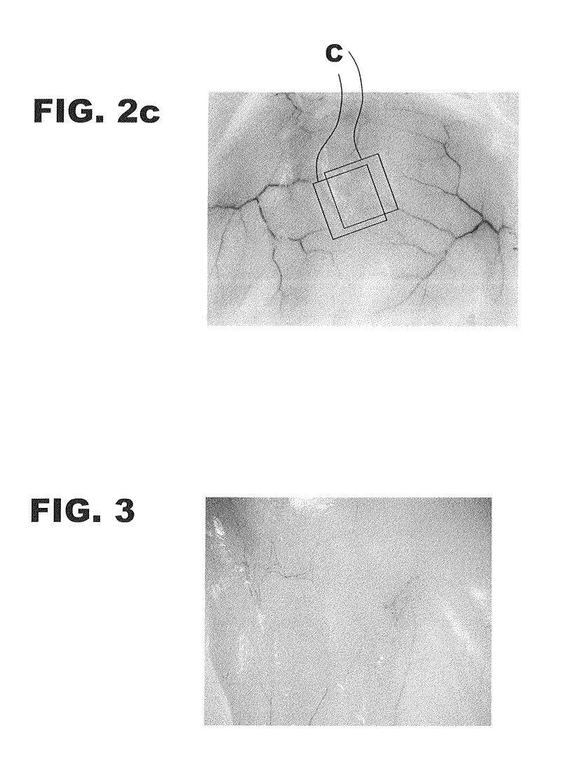

FIG. 3 shows the result. The relatively thin gelatin films B are already substantially resorbed and have lost their structural integrity after 21 days. At the same time, the photograph shows that the newly formed blood vessels, which were observed in the corresponding films after 12 days (see FIG. 2b), have receded again.

This result shows that the angiogenetic effect of the non-porous shaped body is temporary. As resorption of the angiogenesis-promoting substrate progresses, the blood vessels also recede again. However, the resorption speed and, therefore, also the time frame of the angiogenesis can be influenced by the choice of the prescribed degree of cross-linking.

All in all, these tests confirm that in terms of both space and time, the angiogenesis can be stimulated in the human or animal body with the aid of the substrate according to the invention.

1. An angiogenesis-promoting substrate, comprising a non-porous shaped body formed from a gelatin-containing material wherein the non-porous shaped body is insoluble and resorbable under physiological conditions.

2. The substrate in accordance with claim 1, wherein the gelatin-containing material consists predominantly of gelatin.

3. The substrate in accordance with claim 2, wherein the gelatin-containing material consists essentially entirely of gelatin.

4-5. (canceled)

6. The substrate in accordance with claim 1, wherein the gelatin has an endotoxin content, determined in accordance with the LAL test, of approximately 1,200 I.U./g or less.

7. The substrate in accordance with claim 1, wherein the gelatin-containing material is cross-linked.

8. The substrate in accordance with claim 7, wherein the gelatin is cross-linked.

9. The substrate in accordance with claim 7, wherein the gelatin-containing material is cross-linked using formaldehyde.

10. (canceled)

11. The substrate in accordance with claim 1, wherein the non-porous shaped body is a sheet material.

12. The substrate in accordance with claim 11, wherein the sheet material is a film.

13. The substrate in accordance with claim 12, wherein the film has a thickness ranging from approximately 20 to approximately 500 μm.

14. The substrate in accordance with claim 1, wherein the non-porous shaped body is in the form of particles.

15. The substrate in accordance with claim 14, wherein the particles have an average diameter of from approximately 0.1 mm to approximately 5 mm.

16. The substrate in accordance with claim 1, wherein the non-porous shaped body comprises one or more pharmaceutically active substances not based on gelatin.

17. The substrate in accordance with claim 1, wherein the non-porous shaped body is colonized with cells.

18-23. (canceled)

24. A method of promoting angiogenesis in a human or animal body, comprising
providing a substrate comprising a non-porous shaped body formed from a gelatin-containing material wherein the non-porous shaped body is insoluble and resorbable under physiological conditions; and
applying the substrate in or on the human or animal body.

25. The method of claim 24, wherein applying the substrate in or on the human or animal body comprises applying the substrate to a wound.

26. The method of claim 25, wherein applying the substrate to a wound comprises applying the substrate to skin injuries or burns.

27. The method of claim 24, wherein applying the substrate in the human or animal body comprises implanting the substrate into the body.

28. The method of claim 27, further comprising
colonizing the substrate with cells prior to implanting the cell-colonized substrate into the body.
